(19) United States
(12) Patent Application Publication
(10) Pub. No.: US 2023/0069157 A1
(43) Pub. Date: Mar. 2, 2023

(54) METHOD AND SYSTEM FOR DISPLAYING PATIENT OXYGENATION

(71) Applicant: Covidien LP, Mansfield, MA (US)

(72) Inventor: Paul S. Addison, Edinburgh (GB)

(21) Appl. No.: 17/899,179

(22) Filed: Aug. 30, 2022

Related U.S. Application Data
(60) Provisional application No. 63/239,554, filed on Sep. 1, 2021.

(51) Int. Cl. A61B 5/145; A61B 5/00

(52) U.S. Cl. CPC A61B 5/14542; A61B 5/7425; A61B 5/743; A61B 5/746

(57) ABSTRACT

Methods and systems for displaying patient oxygenation for a neonatal and other settings are described, and in particular displaying oxygenation parameters as an overall informative mapping of oxygenation of the patient. In exemplary embodiments, such overall informative mapping of oxygenation includes display of combined oxygenation parameters, including regional oxygen saturation (rSO₂) and arterial oxygen saturation (SpO₂).

### CROSS-REFERENCE TO RELATED APPLICATION

This application claims the benefit of U.S. Provisional Application No. 63/239,554 filed Sep. 1, 2021, the disclosure of which is incorporated herein by reference in its entirety.

### FIELD

The present technology is generally related to a method and system for displaying patient oxygenation for a neonatal and other settings. Pertinent information related to patient oxygenation is displayed on a monitor screen to provide clear and concise quantitative information to a clinician regarding the status of a patient, for example, a neonate. The displayed information may be used to detect instability of a patient's oxygenation status or other conditions related to oxygenation.

### BACKGROUND

The measurement of arterial and regional oxygen saturation is important in the monitoring of many patient groups, and can find particular application for sensitive patient groups, such as for neonatals or for patients with conditions that can result in oxygenation instability.

One technique for monitoring certain physiological characteristics of a patient uses attenuation of light to determine physiological characteristics of a patient. This is used in pulse oximetry, and the devices built based upon pulse oximetry techniques.

A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The amount of light detected or absorbed may then be used to calculate any of a number of physiological parameters, including oxygen saturation (the saturation of oxygen in pulsatile blood, SpO₂), an amount of a blood constituent (e.g., oxyhemoglobin), as well as a physiological rate (e.g., pulse rate or respiration rate) and when each individual pulse or breath occurs. For SpO₂, red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, and/or from curve fitting and/or other interpolative techniques.

Light attenuation is also used for regional or cerebral oximetry. Oximetry may be used to measure various blood characteristics, such as the oxygen saturation of hemoglobin in blood or tissue, the volume of individual blood pulsations supplying the tissue, and/or the rate of blood pulsations corresponding to each heartbeat of a patient. The signals can lead to further physiological measurements, such as respiration rate, glucose levels or blood pressure.

The ease of monitoring arterial and regional oxygen saturation is also important in the medical field, for example for medical practitioners that need the best information possible, as quickly as possible. The present disclosure recognizes that while most physiological parameters are displayed as times series on medical device displays, better techniques for display of patient oxygenation would be beneficial.

Accordingly, there is a need in the art for improved techniques for display of patient oxygenation data in a medical setting.

### SUMMARY

The present disclosure describes methods and systems for displaying patient oxygenation for a neonatal and other settings, and in particular for displaying oxygenation parameters as an overall informative mapping of oxygenation of the patient.

In one exemplary aspect, such overall informative mapping of oxygenation includes display of combined oxygenation parameters, including regional oxygen saturation (rSO₂) and arterial oxygen saturation (SpO₂). In further exemplary aspects, the combined oxygenation parameters further include fractional tissue oxygenation extraction (FTOE) data.

In exemplary aspects, rSO₂ is plotted directly against SpO₂, where the current value of rSO₂ and SpO₂ is shown.

In additional exemplary aspects, the plot additionally shows one or more display lines of equivalent FTOE (equi-FTOE lines).

In further exemplary aspects, the plot displays a history of a predetermined or customizable amount of time for the patient's oxygenation status. In further exemplary embodiments, the plot displays a recent locus of points corresponding to a previous predetermined, selectable, or customizable time period, e.g., the last minute, the last five minutes, the last hour, the last day, etc.

In further exemplary aspects, alarms and/or baselines are also displayed on the plot.

Accordingly, exemplary aspects described herein allow a clinician to easily perceive the relative stability of the patient's oxygen status in the moment or over time and may provide for alarms when thresholds for one or more of rSO₂, SpO₂, and FTOE is crossed.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is an exemplary plot of rSO₂ and SpO₂ (%) over time (in seconds) for a neonate;

FIG. 2 is an exemplary plot of corresponding first and second FTOE signals derived from the signals of FIG. 1;

FIG. 3 shows an exemplary plot with rSO₂ (%) plotted directly against SpO₂ (%) in accordance with aspects of the present disclosure;

FIG. 4 shows another exemplary rSO₂ vs. SpO₂ plot in accordance with aspects of the present disclosure;

FIG. 5 shows another exemplary rSO₂ vs. SpO₂ plot in accordance with aspects of the present disclosure;

FIG. 6 shows another exemplary rSO₂ vs. SpO₂ plot in accordance with aspects of the present disclosure;

FIG. 7 shows another exemplary rSO₂ vs. SpO₂ plot in accordance with aspects of the present disclosure;

FIG. 8 shows another exemplary rSO₂ vs. SpO₂ plot in accordance with aspects of the present disclosure; and

FIG. 9 is a schematic diagram of a method in accordance with aspects of the present disclosure.

### DETAILED DESCRIPTION

As we have noted above, the present disclosure describes methods and systems for displaying patient oxygenation for a neonatal and other settings, and in particular to displaying oxygenation parameters as an overall informative mapping of oxygenation of the patient. In exemplary embodiments, such overall informative mapping of oxygenation includes display of combined oxygenation parameters, including regional oxygen saturation (rSO₂) and arterial oxygen saturation (SpO₂).

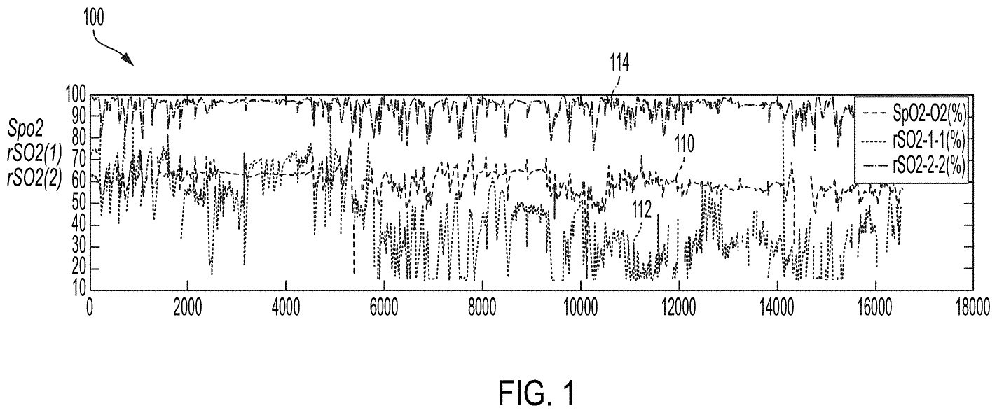

FIG. 1 is an exemplary plot, shown generally at 100, of rSO₂ and SpO₂ (%) over time (in seconds) for a neonate. FIG. 1 shows a time plot of a first neonatal rSO₂ signal, collected from a first neonatal site, at 110, and a second neonatal rSO₂ signal collected a second, different site at 112. FIG. 1 also shows a corresponding time plot of a first neonatal SpO₂ at 114.

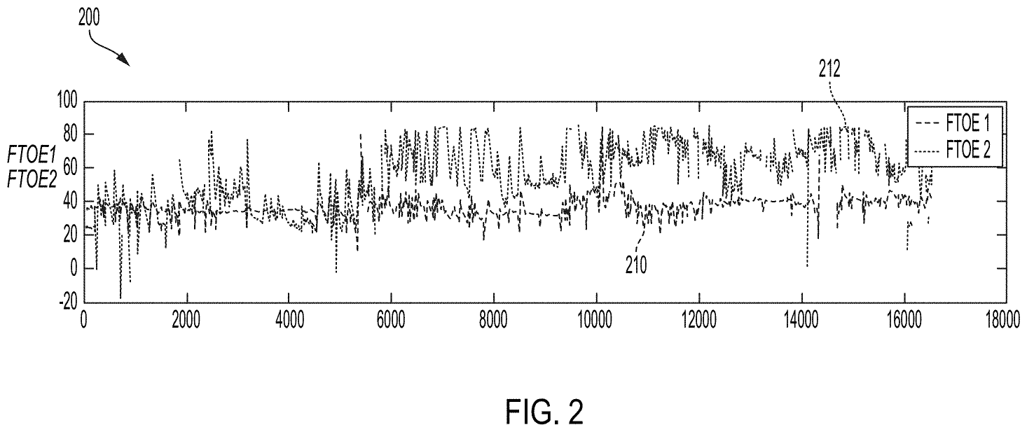

FIG. 2 is an exemplary plot, shown generally at 200, of corresponding first and second FTOE signals, 210 and 212, respectively, derived from the signals of FIG. 1. FTOE is a hybrid signal composed from SpO₂ and rSO₂ as follows:

FTOE=(SpO₂−rSO₂)/SpO₂

FTOE is value calculated to gain more insight into the balance between arterial oxygen delivery to the brain and the brain's oxygen uptake.

As we have noted above, display of rSO₂, SpO₂ and FTOE as a time series on a monitor may have clinical value. However, the present disclosure recognizes that the display of multiple time series may make the situation confusing and that such display of FTOE may obscure differential trending information within the separate SpO₂ and rSO₂ signals. Accordingly, exemplary aspects of the present disclosure provide systems and method that display overall informative mapping of oxygenation as display of combined oxygenation parameters, including regional oxygen saturation (rSO₂) and arterial oxygen saturation (SpO₂). Additionally, in further exemplary embodiments, the combined oxygenation parameters further include fractional tissue oxygenation extraction (FTOE) data.

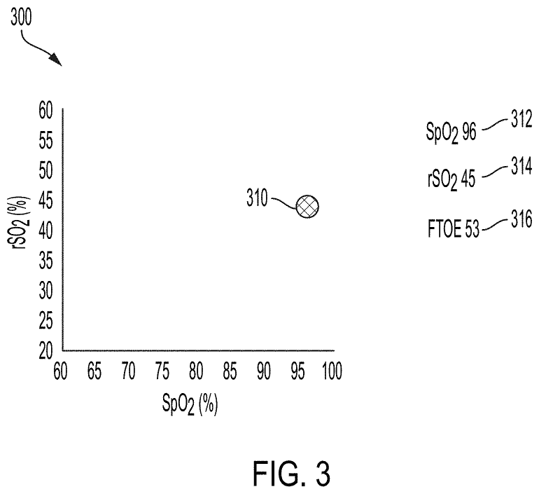

FIG. 3 shows an exemplary plot, generally at 300, with rSO₂ (%) plotted directly against SpO₂ (%), where the current values of rSO₂ and SpO₂ is shown at dot 310. In exemplary embodiments, the display may also show numerical values for one or more of SpO₂ (e.g., at 312), rSO₂ (e.g., at 314) and FTOE (e.g., at 316). In exemplary aspects, as rSO₂ and/or SpO₂ change over time, dot 310 moves around the according to the current values.

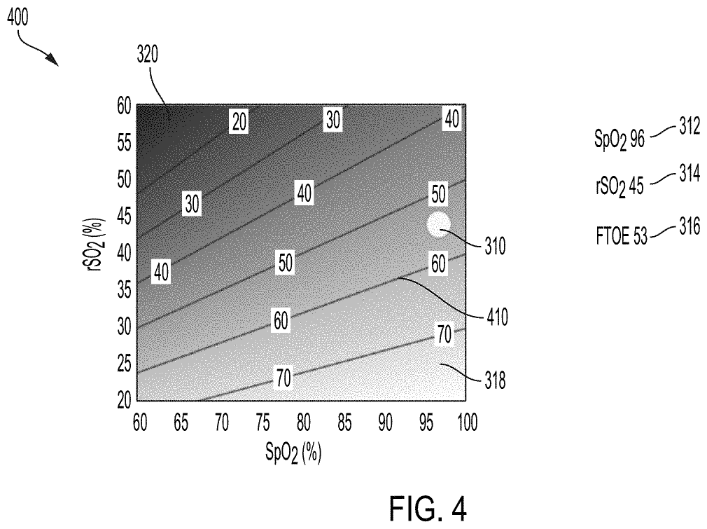

In further exemplary embodiments, shading may also be provided on the display to provide visual context, for example as for the light shading 318 (high FTOE) to dark shading 320 (low FTOE) in FIG. 4.

With further reference to FIG. 4, which shows an exemplary rSO₂ vs. SpO₂ plot generally at 400, further exemplary embodiments may additionally show one or more display lines 410 of equivalent FTOE (equi-FTOE lines) over the plot and relative to dot 310.

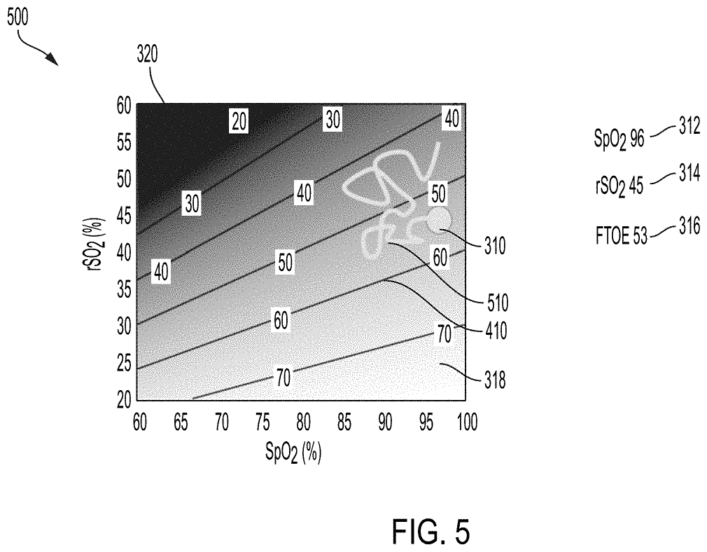

With further reference to FIG. 5, which shows an exemplary rSO₂ vs. SpO₂ plot generally at 500, further exemplary embodiments may additionally display a history of values of rSO₂ and SpO₂, e.g., as history line 510. The amount of historic values for the patient's oxygenation status displayed may be a predetermined or customizable amount of time. In further exemplary embodiments, the plot displays a recent locus of points corresponding to a previous predetermined, selectable, or customizable time period, e.g., the last minute, the last five minutes, the last hour, the last day, etc. We note that as in FIGS. 3-4, FIG. 5 may optionally show current numerical values for rSO₂ (e.g., at 312), SpO₂ (e.g., at 314) and FTOE (e.g., at 316).

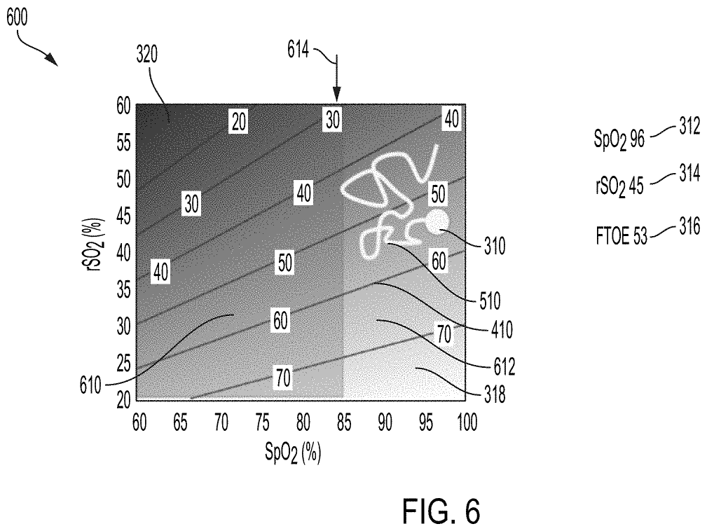

In further exemplary embodiments, alarms and/or baselines are also displayed on the plot. With further reference to FIG. 6, which shows an exemplary rSO₂ vs. SpO₂ plot generally at 600, further exemplary embodiments may additionally display a visual indication for SpO₂ values, e.g., with color or shading 610 showing an area of low SpO₂, area 612 showing normal or high SpO₂, and boundary or line (shown generally at 614) showing an alarm limit for SpO₂. In exemplary embodiments, color can emphasize these areas and/or alarm limits, e.g., with area 610 being red and area 612 being green or blue. This provides a clinician with a clear visual indication of how near the boundary the patient is, particularly if one or more of the dot 310 and the historical path 510 is of a different color or has a high contrast relative to the different displayed SpO₂ areas and/or the displayed SpO₂ alarm limit. Further boundary or line 614 can also have a distinct or high contrast color or appearance.

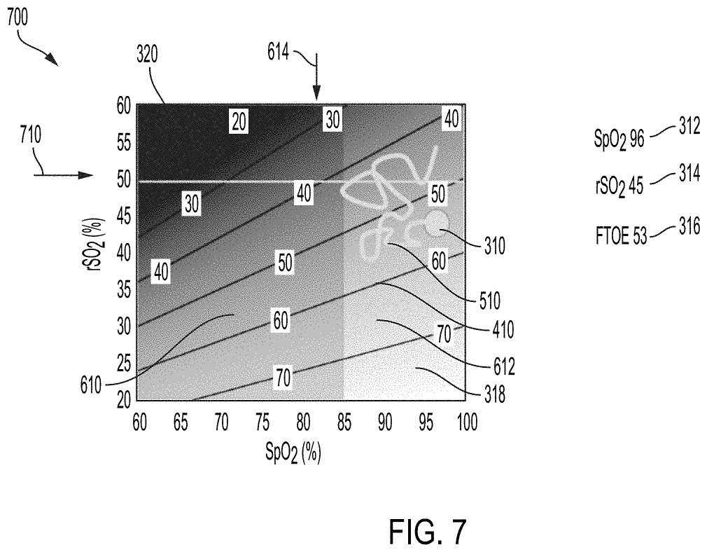

In further exemplary embodiments, additional relevant rSO₂ information is displayed, for example by displaying a baseline for the rSO₂ signal. With further reference to FIG. 7, which shows an exemplary rSO₂ vs. SpO₂ plot generally at 700, further exemplary embodiments may additionally display an rSO₂ baseline, shown generally at 710. In this example, line 710 is shown as a high contrast (e.g., bright yellow) line over the plot, though as with SpO₂ this could be represented by differently shaded or colored areas with the boundary between the two being the desired rSO₂ displayed information. With regard to rSO₂ baseline 710, reference to the dot 310 and/or history path 510 provides a visual indication of how far the patient has moved his/her rSO₂ from a preset baseline (e.g., line 710).

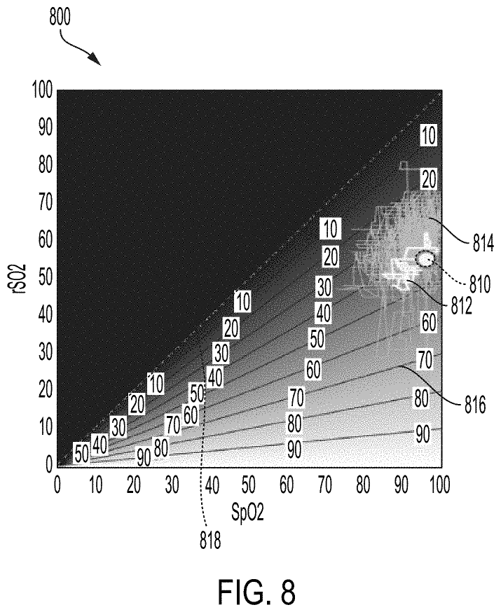

With further reference to FIG. 8, which shows an exemplary rSO₂ vs. SpO₂ plot generally at 800, a current SpO₂−rSO₂ position 810 on the plot is shown by an outlined circle (e.g., a red outlined yellow circle, for emphasized visibility). In this illustrated exemplary embodiment, the most recent 5 minutes of data is depicted by the thicker trace (e.g., a thick yellow trace) 812. The previous 5 hours of data is highlighted by the thinner (e.g., orange) trace 814. It should be recognized that the system or method can be configured with various variations to display aspects, including color, contrast, etc., of the various components. Additionally, the relative amounts of data shown for the history and the way that history is shown can also be preset differently, be customizable, or be selectable by an administrator or user.

Referring again to FIG. 8, the illustrated equi-FTOE lines 816 are also superimposed on the plot. In this illustrated exemplary embodiment, the zero FTOE line 818 is drawn dashed. No data should appear to the right or above of this line as this would mean a higher rSO₂ than SpO₂.

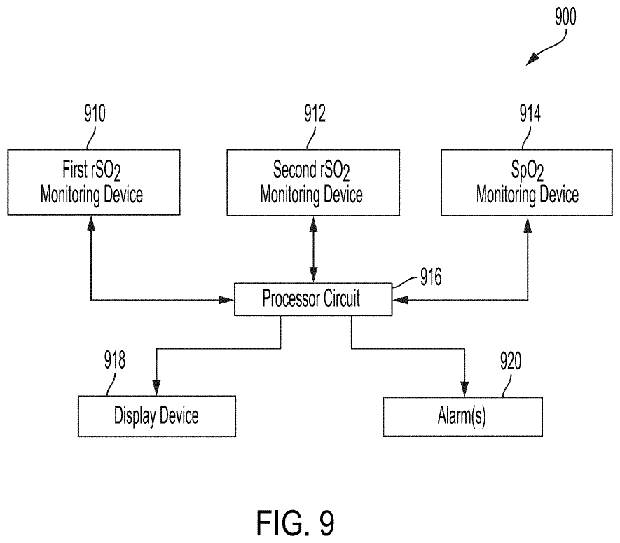

Turning to FIG. 9, a system for displaying oxygenation of a patient is shown generally at 900. As has been described above, various plots and graphing features may be employed to display oxygenation data for a patient on a device, e.g., display device 918. The system further includes a first rSO₂ sensor 910, a second rSO₂ sensor 912 positioned at a different patient location relative to the first rSO₂ sensor 910, and an SpO₂ sensor 914. By way of example, the sensors may be a NELLCOR™ or INVOS™ sensor available from Medtronic (Boulder, Colo.), or another type of oximetry sensor. Further, such sensors may be used in a variety of patient locations, e.g., a patient's fingertip, toe, earlobe, forehead and/or temple, heel, stomach, chest, back, or any other appropriate measurement site.

Referring still to FIG. 9, a processor circuit 910 receives the monitoring data to generate the rSO₂ vs. SpO₂ plots and FTOE information for display on the display device 918. Additionally, the processor circuit 916 can generate or set alarm limits, thresholds or baselines for display on the display device 918 or for activation of one or more alarms 920.

As we have noted, exemplary aspects described herein allow a clinician to easily perceive the relative stability of the patient's oxygen status in the moment or over time and may provide for alarms when thresholds for one or more of rSO₂, SpO₂, and FTOE is crossed. Further, exemplary systems and methods activate or sound an alarm when a threshold for SpO₂, rSO₂ and/or FTOE is crossed, which alarms may include sound, lights, messages, including emails, texts, monitor prompts, etc., or any other mechanism for alerting an individual or monitoring system of a problem.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system for displaying patient oxygenation, comprising:
a first rSO₂ sensor configured to be positioned on a patient at a first location and to provide patient rSO₂ data;
a second rSO₂ sensor configured to be positioned on the patient at a second, different location and to provide patient rSO₂ data;
an SpO₂ sensor configured to be positioned on the patient and to provide patient SpO₂ data; and
a processor configured to plot rSO₂ against SpO₂ and to show a current position on the plot based on said patient rSO₂ and SpO₂ data on a display.

2. A system in accordance with claim 1, wherein the display is also configured to display current numerical values of rSO₂ and SpO₂.

3. A system in accordance with claim 2, wherein the display is also configured to display a current numerical value of FTOE.

4. A system in accordance with claim 1, wherein the rSO₂ against SpO₂ plot additionally shows FTOE lines as an overlay.

5. A system in accordance with claim 4, wherein the FTOE lines as shaded according to high and low values.

6. A system in accordance with claim 1, wherein the rSO₂ against SpO₂ plot additionally shows an SpO₂ threshold or alarm limit, wherein the processor is configured to activate an alarm when the SpO₂ value falls below the threshold or alarm limit.

7. A system in accordance with claim 6, wherein the rSO₂ against SpO₂ plot additionally shades or colors areas above or below the threshold or alarm limit to visually emphasize the relative position of the current position and the SpO₂ threshold or alarm limit.

8. A system in accordance with claim 1, wherein the rSO₂ against SpO₂ plot additionally shows an rSO₂ baseline.

9. A system in accordance with claim 1, wherein the rSO₂ against SpO₂ plot shows the current position on the plot based on said patient rSO₂ and SpO₂ data, as well as a historical trace of past positions.

10. A system in accordance with claim 9, wherein historical trace is customizable as to a length or amount of displayed history, and wherein the characteristics of at least one more current position is emphasized to enhance visual noticeability on the display.

11. A method for displaying patient oxygenation, comprising:
providing a first rSO₂ sensor configured to be positioned on a patient at a first location and to provide patient rSO₂ data;
providing a second rSO₂ sensor configured to be positioned on the patient at a second, different location and to provide patient rSO₂ data;
providing an SpO₂ sensor configured to be positioned on the patient and to provide patient SpO₂ data;
plotting rSO₂ against SpO₂ with a processor;
displaying the rSO₂ against SpO₂ plot on the display as well as displaying a current position on the plot based on said patient rSO₂ and SpO₂ data.

12. A method in accordance with claim 11, further comprising displaying current numerical values of rSO₂ and SpO₂ on the plot.

13. A method in accordance with claim 12, further comprising displaying a current numerical value of FTOE on the plot.

14. A method in accordance with claim 11, further comprising displaying FTOE lines as an overlay on the plot.

15. A method in accordance with claim 14, further comprising displaying shading of FTOE lines on the plot according to high and low values.

16. A method in accordance with claim 11, further comprising showing an SpO₂ threshold or alarm limit on the plot and activating an alarm when the SpO₂ value falls below the threshold or alarm limit.

17. A method in accordance with claim 16, further comprising displaying shading or colored areas above or below the threshold or alarm limit on the plot to visually emphasize the relative position of the current position and the SpO₂ threshold or alarm limit.

18. A method in accordance with claim 11, further comprising display of an rSO₂ baseline on the plot.

19. A method in accordance with claim 1, further comprising showing the current position on the plot based on said patient rSO₂ and SpO₂ data, as well as showing a historical trace of past positions.

20. A method in accordance with claim 19, wherein historical trace is customizable as to a length or amount of displayed history, and wherein the characteristics of at least one more current position is emphasized to enhance visual noticeability on the display.
